(19) United States
(12) Patent Application Publication
(10) Pub. No.: US 2024/0382765 A1
(43) Pub. Date: Nov. 21, 2024

(54) CONDUCTION SYSTEM PACING OPTIMAL OUTPUT SETTING INDICATOR

(71) Applicant: Cardiac Pacemakers, Inc., St. Paul, MN (US)

(72) Inventors: Jessica Burr, Boston, MA (US); Keith L. Herrmann, Minneapolis, MN (US); David L. Perschbacher, Blaine, MN (US)

(21) Appl. No.: 18/652,922

(22) Filed: May 2, 2024

Related U.S. Application Data
(60) Provisional application No. 63/466,865, filed on May 16, 2023.

(51) Int. Cl. A61N 1/37; A61N 1/372

(52) U.S. Cl. CPC A61N 1/3712; A61N 1/37247

(57) ABSTRACT

A method of operating a cardiac rhythm management (CRM) system includes sending a list of electrodes to an ambulatory medical device (AMD) of the CRM system from a programming device for the AMD, the list of electrodes including types of electrodes available to the AMD and position of the electrodes; sending a selection of one or more capture confirming criteria to confirm pacing capture to the AMD; performing, by the AMD, an automatic pacing threshold test for all potential pacing vectors that include the electrodes in the list of electrodes; collecting data for each pace of the pacing threshold test confirmed to capture according to the selected one or more capture confirming criteria; communicating the collected data to the programming device; and presenting the collected data as a trend relative to at least one selected capture confirming criterion and the pacing stimulation energy that resulted in capture.

### CLAIM OF PRIORITY

This application claims the benefit of U.S. Provisional Application No. 63/466,865 filed on May 16, 2023, which is hereby incorporated by reference in its entirety.

### TECHNICAL FIELD

This document relates generally to cardiac rhythm management systems and particularly, but not by way of limitation, to methods, systems, and devices for automatic determination of output settings of a cardiac rhythm management device.

### BACKGROUND

The heart is the center of a person's circulatory system and includes an intrinsic electro-mechanical system for performing two major pumping functions. The left portions of the heart, including a left atrium (LA) and a left ventricle (LV), draw oxygenated blood from the lungs and pump it to body organs to provide the organs with their metabolic need for oxygen. The right portions of the heart, including a right atrium (RA) and a right ventricle (RV), draw deoxygenated blood from the body organs and pump it to lungs where the blood gets oxygenated. These pumping functions result from contractions of the myocardium of the heart. In a normal heart, a sinoatrial (SA) node, the heart's natural pacemaker, generates intrinsic electrical pulses that propagate through an electrical conduction system to various regions of the heart to excite the myocardial tissues of the cardiac muscles. For example, intrinsic electrical pulses originating from the SA node propagate through an atrio-ventricular (AV) node that is between the RA and RV. From the AV node, a specialized intrinsic conduction system is used by the electrical impulses to reach ventricular myocardial tissues, resulting in contraction activities of ventricles. This specialized conduction system includes the His bundle, the right and left conduction bundle branches that extend along the septum between the RV and LV, and the purkinje fibers that contact the ventricular myocardial tissues.

Coordinated delays of the propagations of the intrinsic electrical pulses in a normal electrical conduction system cause the various portions of the heart to contract in synchrony which results in efficient pumping functions. Heart disease can alter the normal intrinsic conduction paths. A blocked or otherwise abnormal electrical conduction can cause the heart to contract dyssynchronously, resulting in poor hemodynamic performance that may diminish the amount of blood supplied to the heart and the rest of the body.

For example, a block in conduction of the electrical pulses in either of the left bundle branch (LBB) or the right bundle branch (RBB) can cause dyssynchrony among the ventricles (RV and LV) of the heart. Blockage of the normal conduction paths can cause intrinsic electrical pulses to conduct along alternate pathways, which can cause one ventricle to contract later with respect to the other ventricle. In such events of cardiac malfunctioning, cardiac pacing therapy can be provided to resynchronize contractions of the ventricles of the heart.

Ambulatory medical devices (AMDs) can be used to provide cardiac pacing therapy to a patient. AMDs, including implantable, subcutaneous, wearable, or one or more other medical devices, etc., can monitor, detect, or treat various conditions, including bradycardia, tachyarrhythmia, cardiac fibrillation, etc. AMDs can be programmable, and a clinician is able to program many different options for pacing parameters. The number of pacing parameters and the introduction of leads with multiple electrodes able to be positioned to several different locations of the heart can create an extensive parameter search space for the clinician to navigate when customizing operation of an AMD to an individual patient. Finding the optimal pacing stimulation parameters may take a lot of time in the clinic for both the clinic staff and the patient.

### SUMMARY

Device-based stimulation therapy can include techniques to reduce the parameter search space for the physician when customizing cardiac pacing stimulation therapy to a particular patient. Example 1 includes subject matter (such as operating a cardiac rhythm management (CRM) system (comprising sending a list of electrodes to an ambulatory medical device (AMD) from a programming device for the AMD, the list of electrodes including types of electrodes available to the AMD and position of the electrodes; sending a selection of one or more capture confirming criteria to confirm pacing capture to the AMD; performing, by the AMD, an automatic pacing threshold test for all potential pacing vectors that include the electrodes in the list of electrodes; collecting data for each pace of the pacing threshold test confirmed to capture according to the selected one or more capture confirming criteria; communicating the collected data to the programming device; and presenting, by the programming device, the collected data as a trend relative to at least one selected capture confirming criterion and the pacing stimulation energy that resulted in capture.

In Example 2, the subject matter of Example 1 optionally includes sending a list of electrodes that includes an electrode positioned in the interventricular septum, collecting pacing capture data for a pacing vector that includes the electrode positioned in the interventricular septum, and presenting a trend of the pacing capture data for the electrode positioned in the interventricular septum and data related to the at least one selected capture confirming criterion.

In Example 3, the subject matter of Example 2 optionally includes using a longer capture detection timing window when testing the electrode positioned at the interventricular septum than when testing a pacing electrode not positioned at the interventricular septum.

In Example 4, the subject matter of one or any combination of Examples 1-3 optionally includes using selectable capture confirming criteria that include a magnitude of one or more heart sounds from a pace confirmed to capture; a width of a far-field QRS complex associated with the pace confirmed to capture, the far-field QRS complex sensed using sensed using a combination of one or more transvenous electrodes and an electrode on the AMD; a time interval from the pace confirmed to capture to a peak amplitude of the far-field QRS complex; and a time interval from the pace confirmed to capture to a sensed electrocardiogram (EGM) of the pace sensed using an electrode combination that delivered the pace.

In Example 5, the subject matter of Example 4 optionally includes sending a selection of one more pacing vectors to the AMD; sending an operating range for the selected one or more capture confirming criteria; recurrently performing automatic pacing threshold tests for the selected vectors according to a schedule and monitoring the selected one or more capture confirming criteria; and setting the one or both of the pacing energy amplitude and pacing energy pulse width for the pacing vectors according to the operating range for the selected one or more capture confirming criteria.

In Example 6, the subject matter of Example 5 optionally includes triggering an alert when an automatic pacing threshold test detects that the selected one or more capture confirming criteria remains outside the operating range for the automatic pacing threshold tests.

In Example 7, the subject matter of one or any combination of Examples 1-6 optionally includes sending a selection of one more pacing vectors to the AMD; sending an operating range for one or both of pacing energy amplitude and pacing energy pulse width for the pacing vectors; and delivering cardiac pacing therapy using the selected pacing vectors.

In Example 8, the subject matter of Example 7 optionally includes recurrently performing automatic pacing threshold tests for the selected vectors according to a schedule; setting the one or both of the pacing energy amplitude and pacing energy pulse width for the pacing vectors according to the recurrent automatic pacing threshold tests; and triggering an alert when the one or both of the pacing energy amplitude and pacing energy pulse width remain outside the operating range for the automatic pacing threshold tests.

In Example 9, the subject matter of one or any combination of Examples 1-8 optionally includes sending a list including can electrodes of the AMD and ring electrodes and tip electrodes of all implantable leads connected to the AMD; and collecting automatic pacing threshold data for all potential pacing vectors that use any combination of the can electrode, ring electrodes, and tip electrodes.

Example 10 includes subject matter (such as an ambulatory medical device) or can optionally be combined with one or any combination of Examples 1-9 to include such subject matter, comprising a cardiac signal sensing circuit configured to sense cardiac signals representative of cardiac activity of a subject when connected to electrodes; a therapy circuit configured to deliver cardiac pacing stimulation energy to the subject when connected to the electrodes; a communication circuit configured to communicate information wirelessly with a separate device; and a control circuit operatively coupled to the cardiac signal sensing circuit, the therapy circuit, and the commination circuit. The control circuit is configured to receive a list of electrodes from the separate device, the list of electrodes including types of electrodes available for delivery of the pacing stimulation energy and position of the electrodes; receive a selection of one or more capture confirming criteria to confirm pacing capture by the pacing stimulation energy; perform an automatic pacing threshold test for all potential pacing vectors that include the electrodes in the list of electrodes; and communicate, to the separate device, pacing capture data for each pace of the pacing threshold test confirmed to capture and capture confirming data of the selected one or more capture confirming criteria.

In Example 11, the subject matter of Example 10 optionally includes a control circuit configured to perform the automatic pacing threshold test to determine optimized pacing stimulation energy to deliver to an electrode positioned in an interventricular septum; and communicate pacing capture data for the interventricular septum and capture confirming data for the selected one or more capture confirming criteria for the interventricular septum.

In Example 12, the subject matter of Example 11 optionally includes a control circuit configured to use a longer capture detection timing window when performing the automatic pacing threshold test using the electrode positioned in the interventricular septum than when performing the automatic pacing threshold test for a pacing vector not including the electrode not positioned in the interventricular septum.

In Example 13, the subject matter of one or any combination of Examples 10-12 optionally includes a heart sound sensing circuit coupled to the control circuit and control circuit configured to collect selected capture confirming data during the automatic pacing threshold test that includes one or more of: a magnitude of one or more heart sounds from a pace confirmed to capture; a width of a far-field QRS complex associated with the pace confirmed to capture, the far-field QRS complex sensed using sensed using a combination of one or more transvenous electrodes and an electrode on the AMD; a time interval from the pace confirmed to capture to a peak amplitude of the far-field QRS complex; and a time interval from the pace confirmed to capture to a sensed electrocardiogram (EGM) of the pace sensed using an electrode combination that delivered the pace.

In Example 14, the subject matter of one or any combination of Examples 10-13 optionally includes a control circuit configured to receive a selection of one or more pacing vectors from the separate device; receive an operating range for the selected one or more capture confirming criteria; recurrently perform maintenance pacing threshold tests for the selected vectors according to a schedule and monitor the selected one or more capture confirming criteria; and adjust the pacing stimulation energy delivered to the pacing vectors according to the operating range for the selected one or more capture confirming criteria.

In Example 15, the subject matter of Example 14 optionally includes a control circuit configured to communicate an alert to the separate device when a maintenance pacing threshold test detects that the selected one or more capture confirming criteria remains outside the operating range for the maintenance pacing threshold test.

In Example 16, the subject matter of one or any combination of Examples 10-15 optionally includes a control circuit configured to receive a selection of one or more pacing vectors from the separate device in response to communicating the pacing capture data to the separate device; receive an operating range for one or both of pacing energy amplitude and pacing energy pulse width for the pacing stimulation energy delivered to the pacing vectors; and deliver the pacing stimulation energy to the selected pacing vectors.

In Example 17, the subject matter of Example 16 optionally includes a control circuit configured to recurrently perform maintenance pacing threshold tests for the selected vectors; set the one or both of the pacing energy amplitude and pacing energy pulse width for the pacing vectors according to the maintenance pacing threshold tests; and communicate an alert to the separate device when the one or both of the pacing energy amplitude and pacing energy pulse width remain outside the operating range for a maintenance pacing threshold test.

Example 18 includes subject matter (such as a programming device for an ambulatory medical device (AMD)) or can optionally be combined with one or any combination of Examples 1-17 to include such subject matter, comprising a communication circuit configured to communicate information wirelessly with the AMD; a user interface; and a programming control circuit operatively coupled to the communication circuit and user interface. The programming control circuit is configured to send a list of electrodes to the AMD that includes types of electrodes available to the AMD to deliver pacing stimulation energy and position of the electrodes; send a selection of one or more capture confirming criteria to confirm pacing capture to the AMD; send a command to the AMD to perform an automatic pacing threshold test for all potential pacing vectors that include the electrodes in the list of electrodes; receive pacing capture data for each pace of the pacing threshold test confirmed to capture and capture confirming data of the selected one or more capture confirming criteria; and present, using the user interface, a trend of pacing capture data relative to at least one selected capture confirming criterion and the pacing stimulation energy that resulted in pacing capture.

In Example 19, the subject matter of Example 18 optionally includes a programming circuit is configured to include an electrode positioned in the interventricular septum in the list of electrodes; receive pacing capture data for a pacing vector that includes the electrode positioned in the interventricular septum; and present a trend of the pacing capture data for the electrode positioned in the interventricular septum and data related to the at least one selected capture confirming criterion.

In Example 20, the subject matter of Example 19 optionally includes a programming circuit configured to receive, via the user interface, a selection of one or more capture confirming criteria to confirm pacing capture by the at least one electrode positioned in the interventricular septum. The capture confirming criteria include one or more of a magnitude of one or more heart sounds from a pace confirmed to capture; a width of a far-field QRS complex associated with the pace confirmed to capture, the far-field QRS complex sensed using sensed using a combination of one or more transvenous electrodes and an electrode on the AMD; a time interval from the pace confirmed to capture to a peak amplitude of the far-field QRS complex; and a time interval from the pace confirmed to capture to a sensed electrocardiogram (EGM) of the pace sensed using an electrode combination that delivered the pace.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example Cardiac Rhythm Management (CRM) system.

FIG. 2 illustrates an example of an ambulatory medical device (AMD) and an environment in which it operates.

FIG. 3 is a block diagram of the electronic circuits of an AMD.

FIG. 4 is a block diagram of an external device that communicates information with an AMD.

FIG. 5 is a flow diagram of a method of operating a CRM system.

### DETAILED DESCRIPTION

Ambulatory medical devices (AMDs) can be used to provide cardiac spacing therapy to a patient. AMDs can include, or be configured to receive physiologic information from, one or more sensors located within, on, or proximate to a body of a patient. Physiologic information of the patient can include, among other things, respiration information (e.g., a respiratory rate, a respiration volume (tidal volume), cardiac acceleration information (e.g., cardiac vibration information, pressure waveform information, heart sound information, endocardial acceleration information, acceleration information, activity information, posture information, etc.); impedance information; cardiac electrical information; physical activity information (e.g., activity, steps, etc.); posture or position information; pressure information; plethysmograph information; chemical information; temperature information; or other physiologic information of the patient.

Conventional right ventricular (RV) pacing therapy provides pacing pulses to the RV via electrodes such as to provide relief to a subject suffering from blockage of normal conduction pathways of the right ventricle. Conduction system pacing (CSP) is the direct pacing of the conduction system of the heart, leading to a more physiological activation of the ventricles alternative to traditional right ventricular pacing. CSP therapy provides pacing pulses at multiple positions within the conduction system (e.g., the His Bundle and the Left Bundle Branch) via electrodes located at these positions, and multiple types of capture can result from pacing at these positions.

The number of pacing electrodes available to an AMD to treat cardiac disease, and the increase in the number of programmable features of AMDs to treat cardiac disease can create an extensive parameter search space for the physician or clinician. The present inventors have recognized, among other things, systems and methods for device-based collection and analysis of data can help reduce the parameter search space.

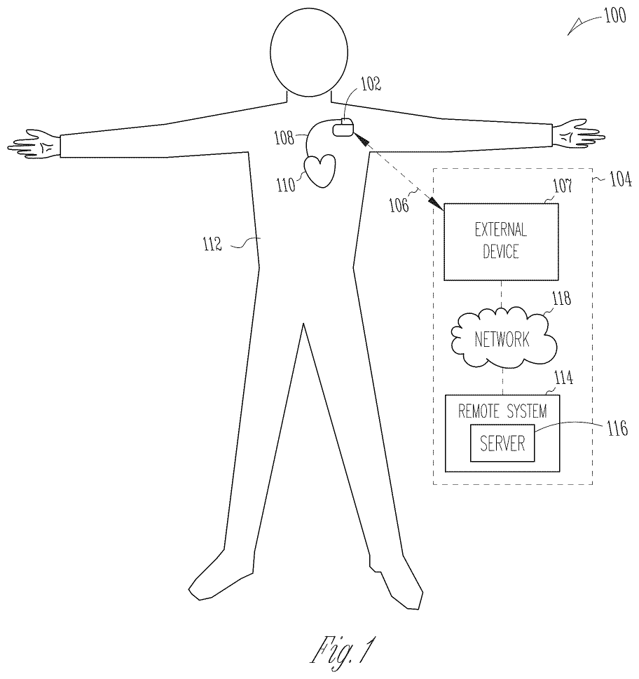

FIG. 1 illustrates portions of an example of a CRM system 100 and portions of an environment in which the CRM system 100 can be used. The CRM system 100 can include an AMD 102 that is implantable, an external system 104, and a communication link such as a telemetry link 106. The AMD 102 can include an electronic unit coupled by a cardiac lead 108, or additional leads, to a heart 110 of a subject 112. Examples of the AMD 102 can include, but are not limited to, pacemakers, pacemaker/defibrillators, cardiac resynchronization devices, cardiac remodeling control devices, and cardiac monitors. In an example, the AMD 102 can be configured to monitor health of the heart 110 and determine one or more abnormalities associated with the heart 110. The AMD 102 can take a necessary action, such as stimulating one or more portions of the heart 110 through the lead 108, to treat the one or more abnormalities.

In an example, the external system 104 can include an external device 107 configured to communicate bi-directionally with the AMD 102 such as through the telemetry link 106. For example, the external device 107 can include a programmer to program the AMD 102 to provide one or more therapies to the heart 110. In an example, the external device 107 can program the AMD 102 to detect presence of a conduction block in a left bundle branch (LBB) of the heart 110 and prevent dyssynchronous contraction of the heart 110 by providing a cardiac resynchronization therapy (CRT) to the heart 110.

In an example, the external device 107 can be configured to transmit data to the AMD 102 through the telemetry link 106. Examples of such transmitted data can include programming instructions for the AMD 102 to acquire physiological data, perform at least one self-diagnostic test (such as for a device operational status), or deliver at least one therapy or any other data. In an example, the AMD 102 can be configured to transmit data to the external device 107 through the telemetry link 106. This transmitted data can include real-time physiological data acquired by the AMD 102 or stored in the AMD 102, therapy history data, an operational status of the AMD 102 (e.g., battery status or lead impedance), and the like. The telemetry link 106 can include an inductive telemetry link or a far-field radio-frequency telemetry link.

In an example, the external device 107 can be a part of a CRM system that can include other devices such as a remote system 114 for remotely programming the AMD 102. In an example, the remote system 114 can be configured to include a server 116 that can communicate with the external device 107 through a telecommunication network 118 such as to access the AMD 102 to remotely monitor the health of the heart 110 or adjust parameters associated with the one or more therapies.

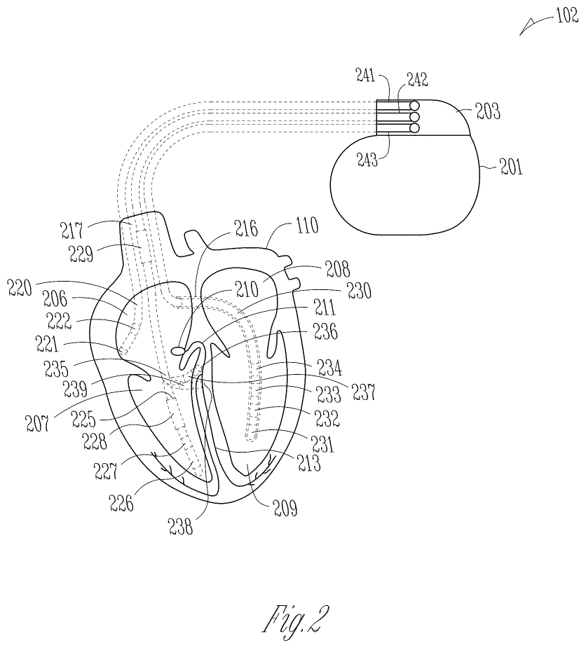

FIG. 2 illustrates an AMD 102 that is implantable and is electrically coupled to a heart 110, such as through one or more leads coupled to the AMD 102 through one or more lead ports, such as first, second, or third lead ports 241, 242, 243 in a header 203 of the AMD 102. In an example, the AMD 102 can include an antenna, such as in the header 203, configured to enable communication with an external system and one or more electronic circuits (e.g., an assessment circuit, etc.) in a hermetically sealed housing (CAN) 201. The AMD 102 illustrates an example ambulatory medical device (or a medical device system) as described herein.

The AMD 102 may include an implantable cardiac monitor (ICM), pacemaker, defibrillator, cardiac resynchronizer, or other subcutaneous AMD or cardiac rhythm management (CRM) device configured to be implanted in a chest of a subject, having one or more leads to position one or more electrodes or other sensors at various locations in or near the heart 110, such as in one or more of the atria or ventricles. Separate from, or in addition to, the one or more electrodes or other sensors of the leads, the AMD 102 can include one or more electrodes or other sensors (e.g., a pressure sensor, an accelerometer, a gyroscope, a microphone, etc.) powered by a power source in the AMD 102. The one or more electrodes or other sensors of the leads, the AMD 102, or a combination thereof, can be configured detect physiologic information from, or provide one or more therapies or stimulation to, the patient.

The AMD 102 can include one or more electronic circuits configured to sense one or more physiologic signals, such as an electrogram or a signal representing mechanical function of the heart 110. In certain examples, the CAN 201 may function as an electrode such as for sensing or pulse delivery. For example, an electrode from one or more of the leads may be used together with the CAN electrode such as for unipolar sensing of an electrogram or for delivering one or more pacing pulses. A defibrillation electrode (e.g., the first defibrillation coil electrode 228, the second defibrillation coil electrode 229, etc.) may be used together with the electrode of the CAN 201 to deliver one or more cardioversion/defibrillation pulses.

The example lead configuration in FIG. 2 include first, second, and third leads 220, 225, 230 in traditional lead placements in the right atrium (RA) 206, right ventricle (RV) 207, and in a coronary vein 216 (e.g., the coronary sinus) over the left atrium (LA) 208 and left ventricle (LV) 209, respectively. The example in FIG. 2 also shows a fourth lead 235 that can be positioned in the RV 207 near the His bundle 211 in the interventricular septum below the sinoatrial (SA) node 210 or positioned in the RV 207 near the left bundle branches 213.

Each lead can be configured to position one or more electrodes or other sensors at various locations in or near the heart 110 to detect physiologic information or provide one or more therapies or stimulation. The first lead 220, positioned in the RA 206, can include a first tip electrode 221 located at or near the distal end of the first lead 220 and a first ring electrode 222 located near the first tip electrode 221. The second lead 225 is positioned in the RV 207 and can include a second tip electrode 226 located at or near the distal end of the second lead 225 and a second ring electrode 227 located near the second tip electrode 226. The third lead 230, positioned in the coronary vein 216 over the LV 209, can include a third tip electrode 231 located at or near the distal end of the third lead 230, a third ring electrode 232 located near the third tip electrode 231, and two additional ring electrodes 233, 234.

The fourth lead 235 is positioned in the RV 207. For simplicity of the Figure, the lead 235 is shown optionally positioned near the His bundle 211 or optionally positioned near the left bundle branches 213, but the system may include a separate fourth lead near the His bundle 211 and a fifth lead near the left bundle branches 213. The fourth lead 235 can include a fourth tip electrode 236 located at or near the distal end of the fourth lead 235 and a fourth ring electrode 237 located near the fourth tip electrode 236. The fourth lead 235 can include a fifth tip electrode 238 located at or near the distal end of the lead and a fifth ring electrode 239 located near the fifth tip electrode 238. The tip and ring electrodes can include pacing/sensing electrodes configured to sense electrical activity or provide pacing stimulation.

In addition to tip and ring electrodes, one or more leads can include one or more defibrillation coil electrodes configured to sense electrical activity or provide cardioversion or defibrillation shock energy. For example, the second lead 225 can include a first defibrillation coil electrode 228 located near the distal end of the second lead 225 in the RV 207 and a second defibrillation coil electrode 229 located a distance from the distal end of the second lead 225, such as for placement in or near the superior vena cava (SVC) 217.

Different CRM devices may include different number of leads and lead placements. For example, some CRM devices are single-lead devices having one lead (e.g., RV only, RA only, etc.). Other CRM devices are multiple-lead devices having two or more leads (e.g., RA and RV; RV and LV; RA, RV, and LV; etc.). CRM devices adapted for His bundle pacing or left bundle branch pacing may use lead ports designated for LV or RV leads to deliver stimulation to the His bundle 211 or left bundle branches 213.

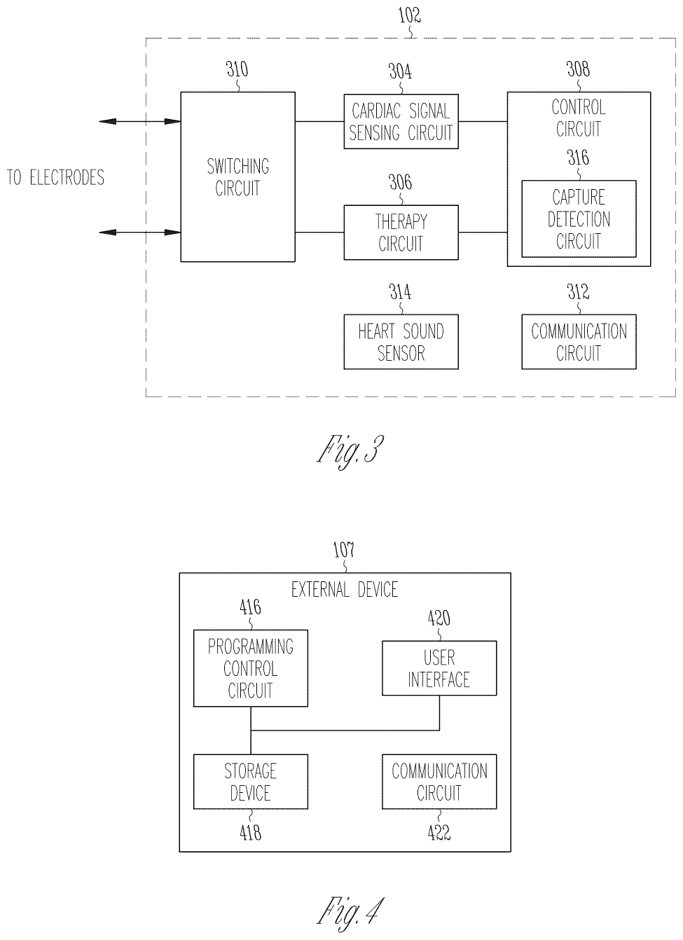

FIG. 3 is a block diagram of portions of electronic circuits of an AMD 102 that is implantable. The AMD 102 can be coupled to multiple implantable electrodes, such as the electrode arrangement described in the example of FIG. 2. The AMD 102 includes a cardiac signal sensing circuit 304, a therapy circuit 306, a heart sound sensor 314, a switching circuit 310, a communication circuit 312, and a control circuit 308. The therapy circuit 306 provides electrical pacing stimulation energy to the heart of the patient when operatively connected to pacing electrodes of the system. The pacing electrodes can include any of the pacing electrodes in FIG. 2, such as electrodes configured placement in or near the RA, RV, LV, His Bundle, or left bundle branches, and an electrode of the CAN.

The cardiac signal sensing circuit 304 includes one or more sense amplifiers to sense one or both of a voltage signal or a current signal at the electrodes. Cardiac electrical information of the patient can be sensed using the cardiac signal sensing circuit 304. Timing metrics between different features in a sensed electrical signal (e.g., first and second cardiac features, etc.) can be determined, such as by the control circuit 308. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc. Far-field cardiac signals can be sensed using the electrode of the CAN.

Cardiac acceleration information of the patient can be sensed using the heart sound sensor 314. Heart sounds are recurring mechanical signals associated with cardiac vibrations or accelerations from blood flow through the heart or other cardiac movements with each cardiac cycle or interval and can be separated and classified according to activity associated with such vibrations, accelerations, movements, pressure waves, or blood flow. Heart sounds include four major features: the first through the fourth heart sounds (S1 through S4, respectively).

The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using heart sound sensor 314 (e.g., an accelerometer or a microphone), providing an output referred to herein as cardiac acceleration information or heart sound information.

The switching circuit 310 is to electrically couple different combinations of the electrodes to the therapy circuit 306 and the cardiac signal sensing circuit 304. The switching circuit 310 can configure any combination of the electrodes into a pacing vector to deliver cardiac pacing stimulation energy or configure any combination of the electrodes into a sensing vector to sense a cardiac signal.

The control circuit 308 may include a digital signal processor, application specific integrated circuit (ASIC), field programmable gate array (FPGA), microprocessor, or other type of processor, interpreting or executing instructions in software or firmware. In some examples, the control circuit 308 may include a state machine or sequencer that is implemented in hardware circuits. The control circuit 308 may include any combination of hardware, firmware, or software. The control circuit 308 includes one or more circuits to perform the functions described herein. A circuit may include software, hardware, firmware or any combination thereof. For example, the circuit may include instructions in software executing on the control circuit 308. Multiple functions may be performed by one or more circuits of the control circuit 308.

The control circuit 308 can include a capture detection circuit 316. The capture detection circuit 316 can automatically determine a pacing threshold for the patient. To determine appropriate pacing stimulation energy, the control circuit 308 initiates delivery of a sequence of pacing pulses to the heart. The sequence may include a successive reduction of the energy of the pacing pulses. A first pacing pulse that will likely induce capture is delivered. The energy of subsequent pacing pulses is reduced (e.g., by a reduction in one or both of pulse amplitude and pulse width) in steps until the capture detection circuit 316 verifies that failure to induce capture has occurred. At this point, the control circuit 308 may trigger a high voltage backup pace to prevent any pause in pacing support.

Alternatively, the sequence may include increasing the energy of the pacing pulses (e.g., by increasing one or both of amplitude and pulse width). A first pacing pulse that is below a threshold likely to induce capture is delivered. The energy of subsequent pacing pulses is increased in steps until the capture detection circuit 316 verifies that capture was induced. Pacing capture can be detected by sensing cardiac signals during a predetermined timing window after the pacing pulse is delivered and looking for a resulting R-wave during the window. An approach for an automatic capture threshold test can be found in Sathaye et al., “Capture Detection with Cross Chamber Backup Pacing,” U.S. Pat. No. 8,948,867, filed Sep. 14, 2006, which is incorporated herein by reference in its entirety.

Information obtained from the automatic pacing threshold test can be used to optimize the pacing stimulation energy used in cardiac pacing therapy. Once the failure to induce capture is detected (e.g., in a step down test) or the inducement of capture is detected (e.g., in a step up test), the changing of the stimulation energy level is continued until confirming the inducement of capture or the failure to induce capture. In a step up test, “confirming capture” can mean that the test continues to step up at energy levels higher than the pacing threshold in order to confirm that capture is stable. An automatic pacing threshold test can be performed for all potential pacing vectors of the electrodes of the system using the therapy circuit 306, cardiac signal sensing circuit 304, switching circuit 310, and capture detection circuit 316.

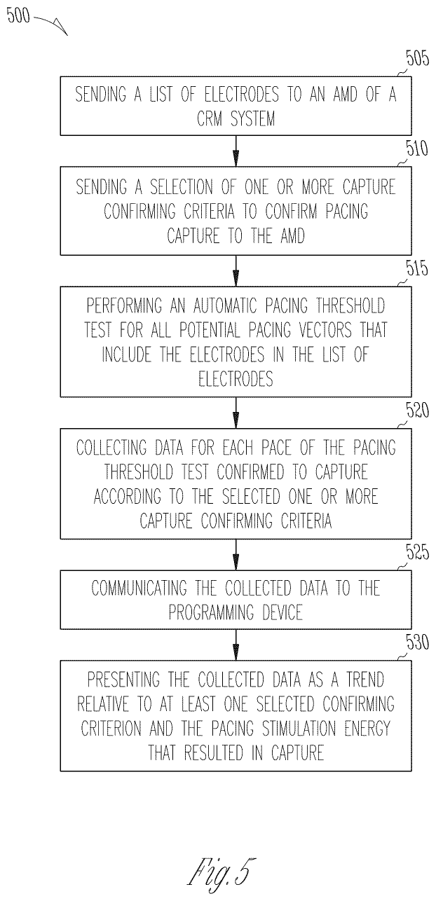

The control circuit 308 uses the communication circuit 312 to communicate information wirelessly with a separate device. FIG. 4 is a block diagram of portions of an example of an external device 107 (e.g., external device 107 of the neurostimulation system 100 in FIG. 1) to communicate with the AMD 102 of FIG. 3. The external device 107 may be a programming device for the AMD 102. Programming device includes a storage device 418, a programming control circuit 416, a user interface 420, and a communication circuit 422. Programming control circuit 416 may be implemented using an application-specific integrated circuit (ASIC) constructed to perform one or more particular functions or a general-purpose circuit programmed to perform such functions. A general-purpose circuit can include, among other things, a microprocessor or a portion thereof, a microcontroller or portions thereof, and a programmable logic circuit or a portion thereof. The storage device 418 may be a memory integral to the programming control circuit 416, or a separate memory device. Communication circuit 422 communicates information wirelessly with the AMD 102 using near-field inductive wireless signals or far-field radio-frequency signals. The programming device can be used to program pacing therapy parameters and other information in the AMD 102.

FIG. 5 is a flow diagram of a method 500 of operating a CRM system that includes an AMD and a programming device. The method 500 helps the clinician navigate the parameter search space to find optimal pacing stimulation parameters for the patient. The method also allows the clinician to configure maintenance testing of the currently programmed neurostimulation parameters when the patient is away from a clinical setting.

At block 505, the programming device sends a list of electrodes to the AMD. The list of electrodes includes the types of the electrodes and the positions of the electrodes. In the example of FIG. 2, the programming device may send a list of electrodes that includes the five tip electrodes, seven ring electrodes, two coil electrodes, any can electrodes, and the corresponding position of the electrodes shown in FIG. 2 (e.g., RA, RV, LV, His bundle, and LBB). This input into the AMD allows the AMD to run automatic pacing threshold tests on the pacing configurations that are available due to the presence of the electrodes. The results of these tests will be used to focus the parameter space for the clinician as an alternative to manual searching by the clinician.

At block 510, the programming device is used to send a selection of one or more capture confirming criteria to confirm pacing capture to the AMD. This input allows the pacing results to be analyzed or assessed in multiple ways. In some examples, the capture confirming criteria can include the magnitude of the S1 heart sound. An increase in the magnitude of the S1 heart sound may indicate a change from pacing non-capture to pacing capture or a stronger depolarization. In some examples, the capture confirming criteria can include the width of the far-field QRS complex in a sensed cardiac signal. A far-field QRS complex refers to sensing the QRS complex using at least one electrode that is not located near the electrodes used to induce capture. For example, pacing capture may be induced using a bipolar electrode configuration and the far-field signal is sensed using a unipolar electrode pair that includes a CAN electrode. A change to a narrower width of a sensed far-field QRS complex may indicate a change from pacing non-capture to pacing capture.

In additional examples, the capture confirming criteria can include the time interval from the pace confirmed to capture to a peak amplitude of the far-field QRS complex, or the time interval from the pace confirmed to capture to an electrocardiogram (EGM) of the pacing capture in which the EGM is sensed an electrode combination or sensing vector that includes at least one electrode concomitant to the pacing vector.

The capture confirming criteria can include further examples. The capture confirming criteria are selectable to tailor the pacing capture analysis to the preferences of the user. The criteria can be selectable using the user interface of the programming device. In some examples, the programming device populates a selection menu with default criteria, and the criteria to confirm capture is updated by the user.

At block 515, when the AMD receives the list of electrodes and the selection for the capture confirming criteria, the AMD performs an automatic pacing threshold test for all of the potential pacing vectors that can include the electrodes in the list. The test or tests may be initiated by a command from the programming device. An electrode can be included in more than one pacing vector in the automatic pacing threshold tests. For example, tip electrode 238 in FIG. 2 located in the interventricular septum can be included in a bipolar pacing vector that includes ring electrode 239 and included in a unipolar pacing vector that includes the can electrode. Ring electrode 239 can be included in the bipolar pacing vector with tip electrode 238 and included in a unipolar pacing vector that includes a CAN electrode. The test may include changing electrode polarity. For example, a first pacing vector may be a bipolar vector including LBB tip electrode 238 and LBB ring electrode 239 with LBB tip electrode 238 as the pacing cathode, and a second pacing vector may include LBB tip electrode 238 and LBB ring electrode 239 with LBB ring electrode 238 as the pacing cathode. A pacing vector may include more than two electrodes. For example, a pacing vector may include LBB tip electrode 238, LBB ring electrode 239, and a CAN electrode.

When the automatic pacing threshold test includes an electrode positioned in the interventricular septum (e.g., one or more of His bundle tip 236, His bundle ring electrode 237, LBB tip electrode 238, and LBB ring electrode 239 in FIG. 2) the capture detection by the AMD may use a longer (or later) timing window to detect the capture to allow for the conduction time from the His bundle to the ventricular myocardium (HV interval) or from the LBB to the ventricular myocardium. Other custom timing windows may be used for the capture detection depending on the conduction time of the specific positions of the electrodes used in the test. Other examples of potential electrode combinations include each electrode of the LV pacing lead, the tip and ring electrodes of a bipolar pacing lead for the RV and RA, and left septum, the tip, ring, and coil electrodes of a defibrillation lead in any position of the heat including RV, left septum, and LBB.

At block 520, the AMD collects data for each pace of the pacing threshold test or tests that is confirmed to capture according to the selected one or more capture confirming criteria. For example, for each pace confirmed to capture, the AMD collects data for one or more of the magnitude of the S1 heart sound, the width of the far-field QRS complex, the time interval from the pace to a peak amplitude of the far-field QRS complex, and the time interval from the pace confirmed to capture to a sensed electrocardiogram (EGM) of the pace, depending on the selections from the user. This data can be collected for each pace confirmed to capture for each potential pacing vector and analyzed per type of pacing electrode and position.

At block 525, the collected data is communicated from the AMD to the programming device. The data can be analyzed by the programming device and at block 530 presented as one or more trends relating the pacing stimulation energy to the selected capture confirming criteria. For example, a trend can be presented on the user interface of the programming device that shows the change in the time interval from the pace confirmed to capture to the peak amplitude of the far-field QRS complex as the pacing stimulation energy is changed (e.g., as one or both of pacing pulse amplitude and pacing pulse width).

The analysis is tailored to the electrodes available to the AMD and only potential vectors for that AMD are included in the analysis. The analysis or analyses selected by the clinician and presented to the clinician is helpful to the clinician in selecting the optimal pacing vector or vectors to treat the patient's condition and setting the optimal pacing stimulation energy or range of pacing stimulation energy for the selected vector or vectors. For example, the trend presented by the programming device may show the time interval from the pace confirmed to capture to the peak amplitude of the far-field QRS complex as 70 milliseconds (70 ms) for a pacing pulse voltage range of 7.5 Volts (7.5V) to 2V, and an interval of 100 ms for a pacing voltage range of 2V to 1V. Based on the trend, the clinician may choose to set the pacing pulse amplitude to higher than 2V.

Note that the analysis is accomplished without the clinician having to specify pacing vectors or pacing stimulation energy. Thus, the parameter space is automatically searched by the device and not manually searched by the clinician. The clinician can then send the selection of the pacing vector or vectors to the AMD. The programming device may sort the pacing vectors according to what the programming device considers the best pacing vector. The clinician can also send an operating range for one or both of pacing energy amplitude and pacing energy pulse width for the selected pacing vectors. The AMD can then deliver the pacing therapy according to the received selections of the clinician.

The clinician can also use the results of the analysis to schedule maintenance tests for pacing capture while the patient is ambulatory and away from a clinical setting. The clinician can select a range for the tolerance of one or more capture confirming criteria that can be the same or different from the originally selected capture confirming criteria. The AMD recurrently performs the maintenance test according to the schedule and monitors the capture confirming criteria for compliance with the indicated range. The clinician may specify the vectors used in the tests to avoid testing of vector poles that showed no capture or resulted in unwanted capture.

In another example, the AMD recurrently measures the one or more capture confirming criteria and initiates a maintenance test when the measurement is outside the indicated range. Based on the results of the maintenance tests, the AMD can automatically change the pacing stimulation energy to ensure stable capture. The AMD may automatically change the pacing stimulation energy to maintain compliance with the selected operating range of the capture confirming criteria. For example, the AMD may change the pacing stimulation energy to keep the time interval from the pace confirmed to capture to the peak amplitude of the far-field QRS complex less than 100 ms.

The AMD may trigger an alert when one or both of the pacing stimulation energy amplitude and pacing stimulation energy pulse width remain outside the operating range for the automatic pacing threshold tests. The AMD may also trigger an alert when the selected operating range of the capture criteria remains outside the selected operating range of the selected capture criteria. The alert may be a flag set inside the AMD that is read by the programming device or a monitoring device in possession of the patient. In some examples, alert is included in a signal sent by the AMD to an external device that communicates with the AMD. In other examples, the medical device itself can provide an audible or tactile alert to warn the patient of the detected change in pacing capture.

The systems, methods and devices described herein provide device-based collection and analysis of data that help reduce the parameter search space when customizing operation of an AMD to treat an individual patient.

### ADDITIONAL DESCRIPTION

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like. In some examples, a carrier medium can carry code implementing the methods. The term “carrier medium” can be used to represent carrier waves on which code is transmitted.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A method of operating a cardiac rhythm management (CRM) system, the method comprising:
sending a list of electrodes to an ambulatory medical device (AMD) of the CRM system from a programming device for the AMD, the list of electrodes including types of electrodes available to the AMD and position of the electrodes;
sending a selection of one or more capture confirming criteria to confirm pacing capture to the AMD;
performing, by the AMD, an automatic pacing threshold test for all potential pacing vectors that include the electrodes in the list of electrodes;
collecting data for each pace of the pacing threshold test confirmed to capture according to the selected one or more capture confirming criteria;
communicating the collected data to the programming device; and
presenting, by the programming device, the collected data as a trend relative to at least one selected capture confirming criterion and the pacing stimulation energy that resulted in capture.

2. The method of claim 1,
wherein the sending the list of electrodes includes sending a list of electrodes that includes an electrode positioned in the interventricular septum;
wherein the collecting data includes collecting pacing capture data for a pacing vector that includes the electrode positioned in the interventricular septum; and
wherein the presenting the trend includes presenting a trend of the pacing capture data for the electrode positioned in the interventricular septum and data related to the at least one selected capture confirming criterion.

3. The method of claim 2, wherein the performing the automatic pacing threshold test includes using a longer capture detection timing window when testing the electrode positioned at the interventricular septum than when testing a pacing electrode not positioned at the interventricular septum.

4. The method of claim 1, wherein the selectable capture confirming criteria include:
a magnitude of one or more heart sounds from a pace confirmed to capture;
a width of a far-field QRS complex associated with the pace confirmed to capture, the far-field QRS complex sensed using sensed using a combination of one or more transvenous electrodes and an electrode on the AMD;
a time interval from the pace confirmed to capture to a peak amplitude of the far-field QRS complex; and
a time interval from the pace confirmed to capture to a sensed electrocardiogram (EGM) of the pace sensed using an electrode combination that delivered the pace.

5. The method of claim 4, including:
sending a selection of one more pacing vectors to the AMD;
sending an operating range for the selected one or more capture confirming criteria;
recurrently performing automatic pacing threshold tests for the selected vectors according to a schedule and monitoring the selected one or more capture confirming criteria; and
setting the one or both of the pacing energy amplitude and pacing energy pulse width for the pacing vectors according to the operating range for the selected one or more capture confirming criteria.

6. The method of claim 5, including triggering an alert when an automatic pacing threshold test detects that the selected one or more capture confirming criteria remains outside the operating range for the automatic pacing threshold tests.

7. The method of claim 1, including:
sending a selection of one more pacing vectors to the AMD;
sending an operating range for one or both of pacing energy amplitude and pacing energy pulse width for the pacing vectors; and
delivering cardiac pacing therapy using the selected pacing vectors.

8. The method of claim 7, including:
recurrently performing automatic pacing threshold tests for the selected vectors according to a schedule;
setting the one or both of the pacing energy amplitude and pacing energy pulse width for the pacing vectors according to the recurrent automatic pacing threshold tests; and
triggering an alert when the one or both of the pacing energy amplitude and pacing energy pulse width remain outside the operating range for the automatic pacing threshold tests.

9. The method of claim 1,
wherein the sending the list of electrodes includes sending a list including can electrodes of the AMD and ring electrodes and tip electrodes of all implantable leads connected to the AMD; and
wherein the collecting data includes collecting automatic pacing threshold data for all potential pacing vectors that use any combination of the can electrode, ring electrodes, and tip electrodes.

10. An ambulatory medical device, the device comprising:
a cardiac signal sensing circuit configured to sense cardiac signals representative of cardiac activity of a subject when connected to electrodes;
a therapy circuit configured to deliver cardiac pacing stimulation energy to the subject when connected to the electrodes;
a communication circuit configured to communicate information wirelessly with a separate device; and
a control circuit operatively coupled to the cardiac signal sensing circuit, the therapy circuit, and the commination circuit; wherein the control circuit is configured to:
receive a list of electrodes from the separate device, the list of electrodes including types of electrodes available for delivery of the pacing stimulation energy and position of the electrodes;
receive a selection of one or more capture confirming criteria to confirm pacing capture by the pacing stimulation energy;
perform an automatic pacing threshold test for all potential pacing vectors that include the electrodes in the list of electrodes; and
communicate, to the separate device, pacing capture data for each pace of the pacing threshold test confirmed to capture and capture confirming data of the selected one or more capture confirming criteria.

11. The device of claim 10, wherein the control circuit is configured to:
perform the automatic pacing threshold test to determine optimized pacing stimulation energy to deliver to an electrode positioned in an interventricular septum; and
communicate pacing capture data for the interventricular septum and capture confirming data for the selected one or more capture confirming criteria for the interventricular septum.

12. The device of claim 11, wherein the control circuit is configured to use a longer capture detection timing window when performing the automatic pacing threshold test using the electrode positioned in the interventricular septum than when performing the automatic pacing threshold test for a pacing vector not including the electrode not positioned in the interventricular septum.

13. The device of claim 10, including:
a heart sound sensing circuit coupled to the control circuit; and
wherein the control circuit is configured to collect selected capture confirming data during the automatic pacing threshold test that includes one or more of:
a magnitude of one or more heart sounds from a pace confirmed to capture;
a width of a far-field QRS complex associated with the pace confirmed to capture, the far-field QRS complex sensed using sensed using a combination of one or more transvenous electrodes and an electrode on the AMD;
a time interval from the pace confirmed to capture to a peak amplitude of the far-field QRS complex; and
a time interval from the pace confirmed to capture to a sensed electrocardiogram (EGM) of the pace sensed using an electrode combination that delivered the pace.

14. The device of claim 13, wherein the control circuit is configured to:
receive a selection of one or more pacing vectors from the separate device;
receive an operating range for the selected one or more capture confirming criteria;
recurrently perform maintenance pacing threshold tests for the selected vectors according to a schedule and monitor the selected one or more capture confirming criteria; and
adjust the pacing stimulation energy delivered to the pacing vectors according to the operating range for the selected one or more capture confirming criteria.

15. The device of claim 14, wherein the control circuit is configured to communicate an alert to the separate device when a maintenance pacing threshold test detects that the selected one or more capture confirming criteria remains outside the operating range for the maintenance pacing threshold test.

16. The device of claim 10, wherein the control circuit is configured to:
receive a selection of one or more pacing vectors from the separate device in response to communicating the pacing capture data to the separate device;
receive an operating range for one or both of pacing energy amplitude and pacing energy pulse width for the pacing stimulation energy delivered to the pacing vectors; and
deliver the pacing stimulation energy to the selected pacing vectors.

17. The device of claim 16, wherein the control circuit is configured to:
recurrently perform maintenance pacing threshold tests for the selected vectors;
set the one or both of the pacing energy amplitude and pacing energy pulse width for the pacing vectors according to the maintenance pacing threshold tests; and
communicate an alert to the separate device when the one or both of the pacing energy amplitude and pacing energy pulse width remain outside the operating range for a maintenance pacing threshold test.

18. A programming device for an ambulatory medical device (AMD), the device comprising:
a communication circuit configured to communicate information wirelessly with the AMD;
a user interface; and
a programming control circuit operatively coupled to the communication circuit and user interface; the programming control circuit configured to:
send a list of electrodes to the AMD that includes types of electrodes available to the AMD to deliver pacing stimulation energy and position of the electrodes;
send a selection of one or more capture confirming criteria to confirm pacing capture to the AMD;
send a command to the AMD to perform an automatic pacing threshold test for all potential pacing vectors that include the electrodes in the list of electrodes;
receive pacing capture data for each pace of the pacing threshold test confirmed to capture and capture confirming data of the selected one or more capture confirming criteria; and
present, using the user interface, a trend of pacing capture data relative to at least one selected capture confirming criterion and the pacing stimulation energy that resulted in pacing capture.

19. The device of claim 18, wherein the programming circuit is configured to:
include an electrode positioned in the interventricular septum in the list of electrodes;
receive pacing capture data for a pacing vector that includes the electrode positioned in the interventricular septum; and
present a trend of the pacing capture data for the electrode positioned in the interventricular septum and data related to the at least one selected capture confirming criterion.

20. The device of claim 19, wherein the programming circuit is configured to receive, via the user interface, a selection of one or more capture confirming criteria to confirm pacing capture by the at least one electrode positioned in the interventricular septum, wherein the one or more capture confirming criteria include one or more of:
a magnitude of one or more heart sounds from a pace confirmed to capture;
a width of a far-field QRS complex associated with the pace confirmed to capture, the far-field QRS complex sensed using sensed using a combination of one or more transvenous electrodes and an electrode on the AMD;
a time interval from the pace confirmed to capture to a peak amplitude of the far-field QRS complex; and
a time interval from the pace confirmed to capture to a sensed electrocardiogram (EGM) of the pace sensed using an electrode combination that delivered the pace.
